(19) United States
(12) Patent Application Publication
(10) Pub. No.: US 2024/0131214 A1
(43) Pub. Date: Apr. 25, 2024

(54) A COMBINED AIRBORNE STERILIZER USING PLASMA AND VHP AND ITS OPERATION METHOD THEREFOR

(71) Applicant: Safeair Disinfect, Inc., Tuscon, AZ (US)

(72) Inventor: Dong Soo KIM, Gyeonggi-do (KR)

(73) Assignee: Safeair Disinfect, Inc., Tuscon, AZ (US)

(21) Appl. No.: 18/257,070

(22) PCT Filed: Dec. 14, 2021
(86) PCT No.: PCT/KR21/18969

§ 371 Date: Jun. 12, 2023

(30) Foreign Application Priority Data
Dec. 14, 2020 (KR) 10-2020-0174128

(51) Int. Cl. A61L 9/22; A61L 2/14; A61L 2/20; A61L 2/26; A61L 9/014; A61L 9/04; A61L 9/12

(52) U.S. Cl. CPC A61L 9/22; A61L 2/14; A61L 2/208; A61L 2/26; A61L 9/014; A61L 9/046; A61L 9/122; A61L 2202/14; A61L 2202/15; A61L 2209/11; A61L 2209/14; A61L 2209/15; A61L 2209/22

(57) ABSTRACT

The present specification relates to a plasma and VHP combined sterilizer comprising a housing. More particularly, the plasma and VHP combined sterilizer comprises: a discharge unit, which is provided at the upper end of a housing and discharges predetermined amount of vaporized hydrogen peroxide; a suction fan, which is provided at one side of the housing and suctions the outer air and discharged hydrogen peroxide; a plasma generation unit including a plurality of plasma modules for generating the outer air and hydrogen peroxide, which are suctioned through the suction fan, as plasma ions through plasma discharge; and a control unit for controlling the overall operation of the plasma and VHP combined sterilizer.

### TECHNICAL FIELD

This specification relates to a fusion sterilizer using plasma and VHP and its operation method, and more particularly, to automatic air and surface disinfection and quarantine devices through plasma and VHP fusion.

### BACKGROUND ART

Various types of disinfection and quarantine devices are being used for disinfection and prevention according to COVID-19 Pandemic. There are three main types: (1) disinfection methods using UV lamps, (2) disinfection and quarantine methods by spraying disinfectant solutions, and (3) disinfection methods using plasma technology.

However, the above three methods using individual technologies have problems as well as their effects and characteristics. First, the air disinfection method using a UV lamp generally has a disinfection effect when the virus distributed in the air is at a UVC lamp wavelength transmission distance by circulating external air in the form of a combination of an air purifier and a short wavelength of UVC (254 nm). In an indoor sealed environment, more than 70% of viruses are distributed not only in the air but also on the floor, ceiling, and walls, so the disinfection effect through air circulation is very limited. Second, when disinfecting indoor air by spraying disinfectant, disinfecting particles must be micro-unit as a prerequisite to disinfect not only air but also surfaces such as walls and ceilings through airflow, and the sprayed particles should be ultrafine particles enough not to wet indoor equipment or equipment.

However, when disinfecting around confirmed COVID-19 patients, the particles of disinfectant sprayed from existing quarantine devices are very large, making it difficult to spread into the air, and it is not suitable for indoor sealing disinfection and quarantine devices. The disinfection effect is also limited to the extent of disinfecting the floor, and there are limitations and problems that cannot be expected to disinfect the space.

In addition, in order to remove residual odors after disinfection, measures such as indoor ventilation and more than 6 hours are required depending on the type of disinfectant. Third, the plasma disinfection method has recently been applied to products in combination with air purifiers, but the basic principle is that plasma ions (OH—, 02-, H+, etc.) are combined with viruses by ionizing atmospheric humidity (H20 molecules) and oxygen molecules (02) through plasma discharge. However, the complex design of this method is very important, such as plasma capacity and module composition, air humidity and air circulation structure, and the structure of the discharge fan after plasma discharge due to its short reduction in ion state, but no specific technology has yet been developed.

### DISCLOSURE

### Technical Problem

Therefore, in order to solve the limitations of existing disinfection methods as described above, it is possible to perform disinfection through VHP, selectively sterilize alone through large amounts of plasma ions, or sequentially in parallel with VHP.

The technical problems to be achieved in the present invention are not limited to the above-mentioned technical problems, and other technical problems not mentioned will be clearly understood by those skilled in the art from the following description.

### Technical Solution

Plasma including housing, VHP convergence sterilizer, a discharge unit installed at the top of the housing and discharging vaporized hydrogen peroxide or other disinfectant of a predetermined size; A suction fan installed on one side of the housing, In the second operation mode, the plasma generating unit is controlled to generate a large amount of plasma ions through the inhaled air and the hydrogen peroxide.

In addition, in this specification, the control unit separates the hydrogen peroxide into water vapor and oxygen, and controls the plasma generator to generate the large amount of plasma ions based on the separated water vapor, oxygen, and the inhaled air.

In addition, in this specification, the first filter is a pre-filter, the second filter is an electrostatic collection type filter, and the third filter is a carbon filter.

In addition, in the present specification, the predetermined size is characterized by 40 microns.

In addition, this specification is characterized by the inclusion of four more support rods connected to each of the above housings and spaced apart from each other at predetermined intervals.

In addition, in this specification, each of the four support rods is characterized by a cover that can be opened and closed at the top and bottom of the support rod.

In addition, in this specification, the control unit controls to change from the first operation mode to the second operation mode when the external humidity amount is greater than the threshold value.

In addition, in the present specification, the diameter of the support rod is the same as the diameter of the discharge part.

In addition, the plurality of plasma modules in this specification include a first plasma module, a second plasma module, and a third plasma module, _and the first plasma module, the second plasma module, and the third plasma module are spaced apart from each other at predetermined intervals.

The operation method of the fusion sterilizer includes a hydrogen peroxide emission step for discharging hydrogen peroxide particles of a predetermined size, an external air and hydrogen peroxide inhalation step for inhaling hydrogen peroxide and external air.

The method may further include measuring the humidity of the space due to hydrogen peroxide discharged from the hydrogen peroxide discharging step and stopping hydrogen peroxide discharging step when the humidity measured in the humidity determining step is greater than or equal to.

### Advantageous Effects

This specification has the effect of thoroughly preventing the spread of infections of viruses such as COVID-19 through a double disinfection process that primarily disinfects through VHP through multiple modes, secondarily disinfecting through large amounts of plasma ion generation, and removing residual disinfectants after disinfection.

### DESCRIPTION OF THE DRAWINGS

FIG. 1 is a perspective view illustrating an example of a plasma and a VHP convergence sterilizer proposed in the present specification.

FIG. 2 is a diagram illustrating an example of an exploded perspective view of a discharge part of a plasma and a VHP fusion/composite sterilizer.

FIG. 3 shows an example of an exploded perspective view of a plasma and a VHP convergence sterilizer.

FIG. 4 is a perspective view illustrating an inner side of a support rod of a plasma and a VHP convergence sterilizer.

FIG. 5 is a diagram illustrating an example of an internal configuration diagram of a plasma and a VHP convergence sterilizer.

FIG. 6 is a diagram illustrating an example of an internal structure of a plasma and a VHP convergence sterilizer.

FIG. 7 is a diagram illustrating an example of an internal block diagram of a plasma and a VHP convergence sterilizer proposed in the present specification.

FIG. 8 is a flowchart illustrating an example of a method of operating a plasma and a VHP convergence sterilizer proposed in the present specification.

### MODE FOR INVENTION

It should be noted that the technical terms used in the present specification are used only to describe specific embodiments and are not intended to limit the idea of the technology disclosed in the present specification. In addition, the technical terms used in this specification should be interpreted as generally understood by those with ordinary knowledge in the field to which the technology disclosed herein belongs, not too comprehensive, or too reduced. In addition, if the technical term used in this specification is a false technical term that does not accurately express the idea of the technology disclosed in this specification, it should be replaced with a technical term that can be correctly understood by a person with ordinary knowledge in the field. In addition, the general terms used in this specification should be interpreted as defined in advance or in context before and after, and should not be interpreted in an excessively reduced sense.

Terms including ordinal numbers, such as first, second, and the like, used in the present specification may be used to describe various elements, but the elements should not be limited by the terms. The terms are used only for the purpose of distinguishing one component from another component. For example, a first component may be referred to as a second component without departing from the scope of the present invention, and similarly, a second component may be referred to as a first component.

Throughout the specification, when a part is “connected (connected, contacted, coupled)” with another part, this includes not only “directly connected” but also “indirectly connected” with another member in between.

In this specification, it should be understood that the term “including” or “have” is intended to specify that there is a feature, number, stage, operation, component, part, or a combination of them described in the specification.

Hereinafter, embodiments disclosed in the present specification will be described in detail with reference to the accompanying drawings, but the same or similar components regardless of the reference numerals will be given the same reference numerals and repeated descriptions thereof will be omitted.

In addition, in describing the technology disclosed in this specification, if it is determined that a specific description of the related known technology may obscure the gist of the technology disclosed in this specification, the detailed description is omitted. In addition, it should be noted that the attached drawings are only for easy understanding of the ideas of the technology disclosed in this specification, and should not be interpreted as limiting the ideas of the technology by the attached drawings.

Plasma and VHP Combined Airborne Sterilizer

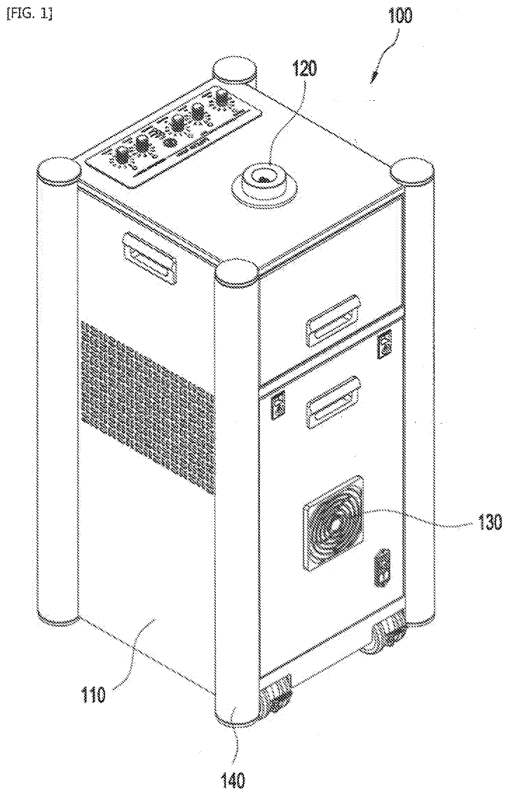

FIG. 1 is a perspective view illustrating an example of a plasma and a VHP convergence sterilizer proposed in the present specification.

As described in FIG. 1, the plasma and VHP convergence sterilizer (100), consist of a housing (110), an exhaust (120) installed at the top of the housing above, a suction fan (130) installed in the housing above, a filter (150) with four support rods (140), and a plasma generator (160) installed in the housing above (110).

Since the components illustrated in FIG. 1 are not essential, a plasma and VHP convergence sterilizer with more or less components may be implemented.

The above plasma and VHP convergence sterilizer operates in three operating modes, and has a great effect on preventing and preventing the spread of infection due to COVID-19 recently through the release of a large amount of plasma ions.

In other words, the above convergence sterilizer is primarily disinfected through vaporized hydrogen peroxide (VHP) or other disinfectants {first operation mode}, and secondary disinfection (second operation mode) through plasma discharge.

The first operation mode of the convergence sterilizer generates VHP or other disinfectant particles of a predetermined size (e.g., 40 microns) through the discharge part of the convergence sterilizer, thereby primarily disinfecting a large space in a short time. For example, convergence sterilizer may disinfect a space based on 1,500 ft² in the first operation mode in 50 minutes.

The second operation mode, that is, the plasma automatic disinfection mode, may be secondary disinfected by inhaling a large amount of VHP or other disinfectant particles generated in the first operation mode into plasma and VHP convergence sterilizer and generating large plasma ions (e.g., three or four plasma modules).

The amount of plasma ions generated through the plurality of plasma modules may be 70 million/cm3.

In addition, the large-capacity plasma ions may include 03-, 02-, HO2-, HO3-, HO4-, 0-, HO—, and the like.

In addition, the remaining residual disinfectant (H2O2 or other disinfectant) can be quickly removed in this process, and the air cleaning function can be automatically performed through the filter part of the air purifier above.

The filter unit may include a pre-filter, an electrostatic collection type filter, and a carbon filter, and a detailed description of each filter will be described later.

In addition, the above plasma and VHP convergence sterilizer may be equipped with a large capacity (e.g., 15 L) disinfectants storage tank, and may be used for about five days at a time (in a 1,500 ft² reference space) using 40 microns of fine particles.

In addition, the above convergence sterilizer is designed b y applying stainless steel 316 materials that do not erode in consideration of disinfectants and quarantine environments. Therefore, users can use various types of disinfectant solutions (e.g., H2O2, NaDCC, etc.), making them competitive in maintenance costs.

In addition, the above plasma and VHP convergence sterilizer has an internal slide structure, so it can easily inject chemical disinfectants and is designed to have convenience in the event of A/S.

In addition, the above plasma and VHP convergence sterilizer has the advantage of being easy to move to the place of use by having a medical wheel at the bottom of the above plasma and VHP convergence sterilizer has the advantage of being easy to move. Therefore, the above plasma and VHP convergence sterilizer can be used in various places such as schools, religious facilities, government offices, hotels, restaurants, banks, and health clubs, and is designed with a high-quality design and competitive compared to other products.

Internal Structure and Function of Plasma and VHP Combined Airborne Sterilizer

Hereinafter, the internal structure and plasma and VHP convergence sterilizer proposed in the present specification will be described in more detail with reference to FIGS. 1 to 6.

As previously seen, plasma and VHP convergence sterilizer (100) generates VHP or other disinfectant particles of a predetermined size through the discharge unit in the first operation mode, inhales external air (02) and hydrogen peroxide into the housing in the second operation mode.

The first operation mode and the second operation mode may be set by a user or may be automatically performed according to a predetermined condition.

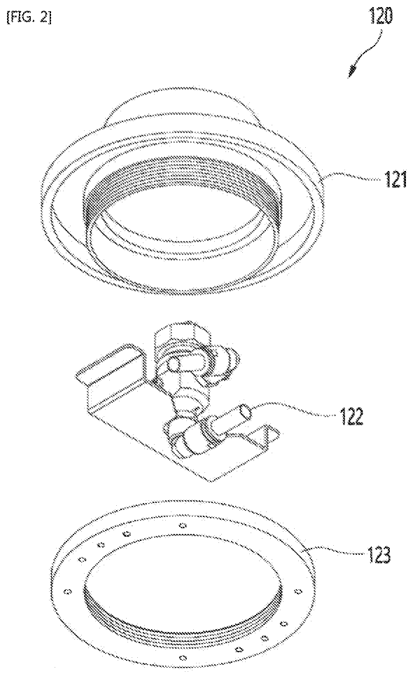

Referring to FIG. 2, the discharge unit (120) includes a discharge cover (121), a spray module (122), and a connection ring (123). FIG. 2 is a diagram illustrating an example of an exploded perspective view of the discharge part of the air purifier.

The emission part is installed (or formed) at the top of the housing of the air purifier and emits vaporized hydrogen peroxide (VHP) of a predetermined size (e.g., 40 microns).

The injection module (122) of the discharge part includes two injection holes, and VHP particles having a predetermined size are discharged through each injection hole. Each of the injection ports may include a injection nozzle, a injection hole, and the like.

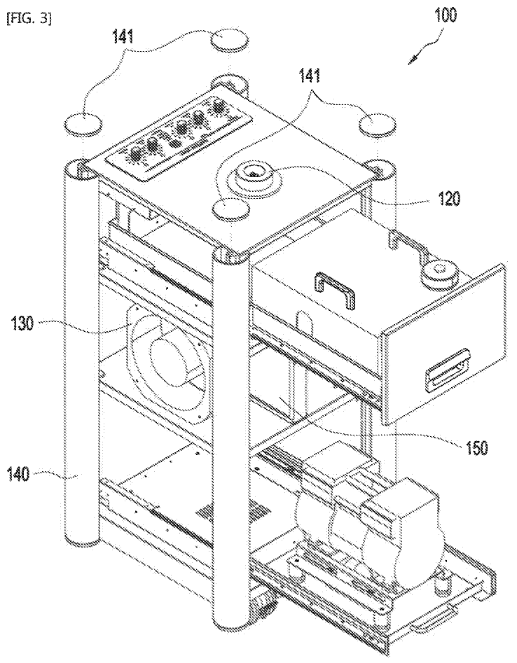

FIG. 3 shows an example of an exploded perspective view of a plasma and VHP convergence sterilizer.

Referring to FIG. 3, the suction fan (130) is installed in the housing of the plasma and VHP convergence sterilizer and inhales external air and the discharged hydrogen peroxide.

The four support rods (140) serve to support the plasma and VHP convergence sterilizer, and each support rod includes an openable cover at the top and bottom of the support rod. The housing of the plasma and VHP convergence sterilizer may be separated in an upward or downward direction by separating the covers of the support rods. The diameter of the support rod may be the same as the diameter of the discharge part, and may be, for example, 70 mm. The characteristic of the structure of the support rod cap (141) has the advantage of being easily separated and dismantled without bolting to the main body.

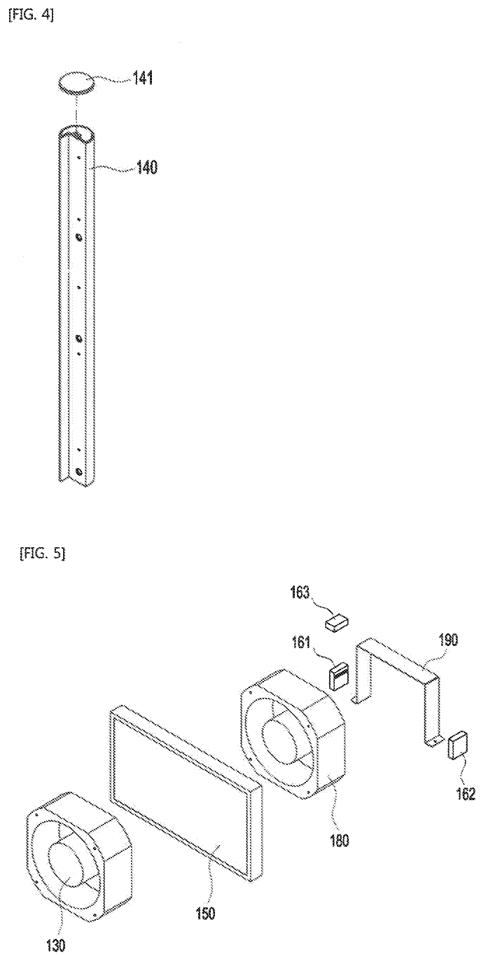

As described in FIG. 4, the inside of the support rod (140) is depressed so that the square edge is inserted, and a number of plates constituting the housing (110) may be fixed to the support set inside the support rod (140) while the support rod cap (141) is separated during the assembly process.

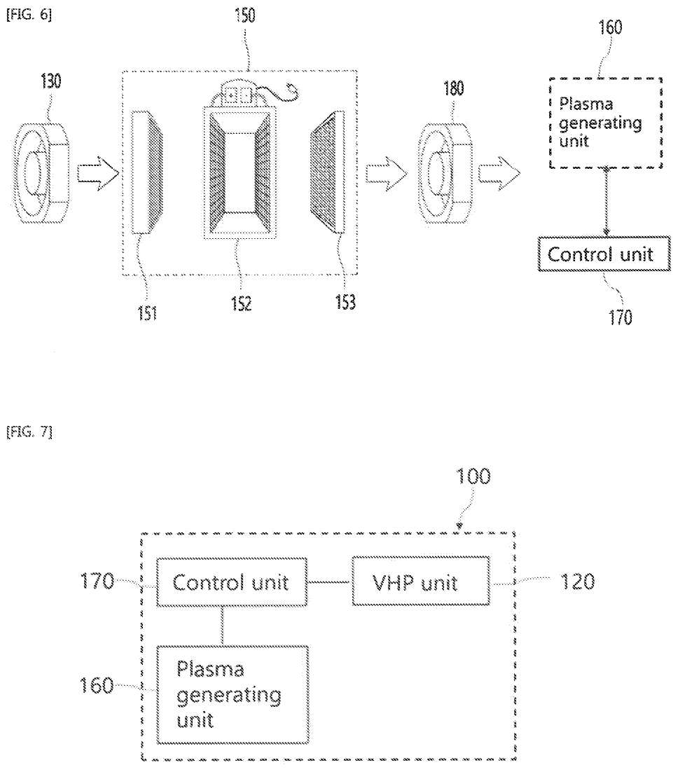

The filter unit (150) is installed behind the suction fan and includes a first filter, a second filter, and a third filter. FIG. 6 is a diagram illustrating an example of an internal structure of a plasma and VHP convergence sterilizer.

Referring to FIG. 5, the first filter (151) may be expressed as a pre-filter, and serves to primarily remove foreign substances with somewhat large particles such as hair, fiber dust, yellow dust, and living dust among foreign substances inhaled from the outside.

The second filter (152) may be represented by a plasma filter and serves to remove fine foreign substances such as ultrafine dust, collection miles, and COVID-19 viruses in the air that have been primarily removed using an electrostatic collection method.

More specifically, the second filter is an electric dust collector using plasma discharge, which generates plasma by applying voltage to electrodes and removes fine dust by electrostatic dust collection by making fine dust negative.

The third filter (153) may be expressed as a carbon filter and serves to remove living odor, cigarette odor, substance that causes birdhouse syndrome, harmful gas, smog harmful substance, etc.

The plasma generator (160) is installed behind the filter unit and includes a plurality of plasma modules.

In addition, each of the multiple plasma modules in the epithelial plasma generator generates a large amount of plasma ions through plasma discharge for the inhaled air and hydrogen peroxide.

The plurality of plasma modules may include three plasma modules, that is, a first plasma module (161), a second plasma module (162), and a third plasma module (163).

At this time, the first plasma module, the second plasma module, and the third plasma module may be spaced apart from each other by a predetermined distance and disposed in a ‘ [symbol] ’ shape on the left, right, and upper ends of the ‘ [symbol] ’ support plate 190 of FIG. 4. FIG. 4 is a diagram illustrating an example of a part of an internal configuration diagram of plasma and VHP convergence sterilizer.

Preferably, the first plasma module (161) and the second plasma module (162) may be disposed to face each other.

That is, plasma and VHP convergence sterilizer proposed in this specification is equipped with multiple plasma modules, and plasma discharge occurs in each plasma module, so a large amount of plasma ions can be generated. In addition, the arrangement of the three plasma modules shown in FIG. 4 (in the form of ‘ [symbol] ’) may be an optimal structure to generate a large amount of plasma ions.

Therefore, in order to generate a large amount of plasma ions, it can be solved by increasing the number of plasma modules included in the plasma generator or by increasing the power of the plasma generator to increase plasma discharge efficiency.

The discharge fan (180) is installed in the housing, may be disposed in front or rear, and in this embodiment, is installed in front of the plasma generation unit. The discharge fan may discharge a large amount of plasma ions generated through the plasma generator to the outside.

The control unit (170) can control the overall operation of the plasma and VHP convergence sterilizer and is functionally connected to the discharge unit (120) and the plasma generation unit (160). In addition, the control unit controls the plasma generator to generate a large amount of plasma ions through plasma discharge for the inhaled air (H2O, 02, etc.) and the hydrogen peroxide (H2O2).

In addition, the above control unit controls the above hydrogen peroxide or other disinfectant so that micro particles can be diffused into the air and circulated to generate the above large amount of plasma ions in the inhaled air and micro-disinfection particles.

In addition, the control unit controls the emission unit to stop the discharge of hydrogen peroxide when the external humidity is detected to be greater than the threshold value, and controls the plasma generator to generate the plasma ions through the plasma discharge.

The threshold value may be a preset value or may be set to be changeable by a user.

For example, the amount of external humidity may increase as the content of hydrogen peroxide vaporized through the discharge part of plasma and VHP convergence sterilizer increases, and if the threshold is set to be changeable by the user, it may change according to the change in external humidity.

Internal Block Diagram of Plasma and VHP Combined Airborne Sterilizer

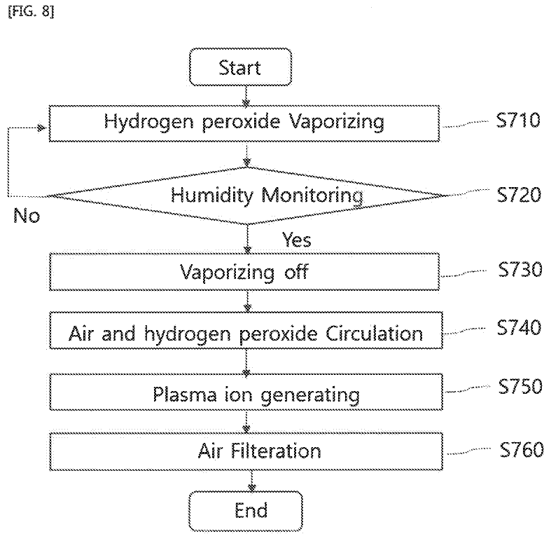

FIG. 7 is a diagram illustrating an example of an internal block diagram of a plasma and a VHP convergence sterilizer proposed in the present specification.

The plasma and VHP convergence sterilizer (100) may include a control unit (170), a vaporizing unit (120), and a plasma generation unit (160).

The components shown in FIG. 6 are not essential, and thus an air purification function having more or less components may be implemented.

The above control unit (170) refers to a module that is functionally connected to the above vaporizing unit and the above plasma generator to control the overall operation of the above plasma and VHP convergence sterilizer.

The control unit may be referred to as an application processor (AP), a processor, a control module, a controller, a microcontroller, a microprocessor, a pressure sensor, or the like, and the processor may be implemented by hardware, firmware, software; or a combination thereof.

The control unit may include an application-specific integrated circuit (ASIC), another chipset, a logic circuit, and/or a data processing device.

The discharge unit (120) generates 40 micron or less VHP or other disinfectant particles to the outside, and can be operated in the first operation mode under the control of the control unit.

The plasma generator 160 generates a large amount of plasma ions (03-, 02-, HO2-, HO3-, HO3-, HO4-, 0-, HO—, OH—, etc.) through plasma discharge for hydrogen peroxide vaporized through the emission unit as well as external air. The plasma generator may be operated in a second operation mode under the control of the controller.

In addition, the plasma and VHP convergence sterilizer (100) may further include a memory and a wireless communication unit.

The memory may store a program for an operation of the controller, and may temporarily store input/output data. The memory is a medium for storing various information of a device, and may be connected to the control unit or the processor to store programs, applications, general files, and input/output data for the operation of the control unit or the processor.

Flash memory type, hard disk type, multimedia card micro type, card type memory (e.g., SD or XD memory), random access memory (RAM), static random access memory (SRAM), RAM (Static Random Access Memory), Reversible (REM) (Electronic) memory (REM), At least one type of storage medium of the optical disk may be included.

The wireless communication unit may include one or more modules that enable wireless communication between the plasma and the wireless communication system of the VHP convergence sterilizer.

That is, the wireless communication unit may include a short-range communication module, a mobile communication module, and a wireless Internet module.

Operation Method of Plasma and VHP Combined Airborne Sterilizer

FIG. 8 is a flowchart illustrating an example of a method of operating a plasma and VHP combined airborne sterilizer proposed in the present specification.

The plasma and VHP convergence sterilizer operation of this embodiment includes a hydrogen peroxide vaporizing stage (S710), a humidity monitoring stage (S720), a hydrogen peroxide vaporizing stop stage (S730), an external air and hydrogen peroxide inhalation stage (S740), a filtering stage (S750, and a plasma generating stage (S760).

In the hydrogen peroxide vaporizing step (S710), VHP nano particles having a predetermined size are vaporized through an vaporized nozzle installed at an upper end of the housing in a first operation mode of the plasma and VHP combined airborne sterilizer and vaporized to the outside.

In the humidity monitoring step (S720), it is determined whether the controller (170) reaches the set humidity value. When the set humidity value is reached, hydrogen peroxide vaporizing stop step (S720) is performed, and hydrogen peroxide vaporizing is stopped. And if the set humidity value is not reached, the hydrogen peroxide vaporizing (S710) continues.

In the external air and hydrogen peroxide inhalation stage (S740), the above plasma and VHP combined airborne sterilizer inhale external air and hydrogen peroxide vaporized through the above exhaust through a suction fan.

In the filtering stage (S750), the air purifier filters the material containing the inhaled air and hydrogen peroxide introduced through the suction fan through the filter unit.

In the plasma generation stage (S760), the air purifier discharges plasma and generates a large amount of plasma ions through each plasma module included in the plasma generation unit.

Meanwhile, the generated plasma ions are discharged to the outside by the discharge fan.

The embodiments described above are those in which the components and features of the present invention are combined in a predetermined form. Each component or feature shall be considered optional unless otherwise stated. Each component or feature may be implemented in a form that is not combined with other components or features. It is also possible to form an embodiment of the present invention by combining some components and/or features. The order of operations described in embodiments of the present invention may be changed. Some configurations or features of some embodiments may be included in other embodiments, or may be replaced with corresponding configurations or features of other embodiments. It is obvious that claims that do not have an explicit citation relationship in the scope of the patent claim can be combined to form an embodiment or included as a new claim by correction after the application.

Embodiments according to the present invention may be implemented by various means, for example, hardware, firmware, software, or a combination thereof. In the case of hardware implementation, an embodiment of the present invention may be implemented by one or more application specific integrated circuits (ASICs), digital signal processors (DSPs), digital signal processing devices (DSPs), programmable logic devices (PLDs), FPGAs (field), microcontroller, etc.

In the case of implementation by firmware or software, an embodiment of the present invention may be implemented in the form of a module, procedure, function, or the like performing the functions or operations described above. The software code may be stored in a memory and driven by a processor. The memory may be located inside or outside the processor to exchange data with the processor by various known means.

It is obvious to those skilled in the art that the present invention may be embodied in other specific forms without departing from the essential features of the present invention. Therefore, the detailed description described above should not be construed as restrictiv12: in all respects and should be considered as illustrative. The scope of this invention should be determined by reasonable interpretation of the appended claims, and all changes in the equivalent scope of this invention are contained in the scope of this invention.

1. A plasma and vaporized hydrogen peroxide (VHP) combined airborne sterilizer comprising:
a plasma generator having one or more plasma modules to generate plasma ions through plasma discharge to external air and hydrogen peroxide introduced through a suction fan; and
a control unit to control a first plasma module of the plasma generator, the first plasma module generates plasma ions in the upper part of a housing, wherein the plasma and VHP combined airborne sterilizer is configured to control the plasma generator to generate a large amount of plasma ions through the inhaled air and the hydrogen peroxide in a second operation mode.

2. The plasma and VHP combined airborne sterilizer of claim 1, the control unit controls an exhaust unit to vaporize hydrogen peroxide into nano and micro particles in a first operation mode, the suction fan to inhale hydrogen peroxide and air circulated from the outside, and the plasma generator to generate a large amount of plasma ions.

3. The plasma and VHP combined airborne sterilizer of claim 1, further comprising:
a first filter is a pre-filter, a second filter is an electrostatic collection type filter, and a third filter is a carbon filter.

4. The plasma and VHP combined airborne sterilizer of claim 1, wherein a predetermined particle size is 40 microns.

5. The plasma and VHP combined airborne sterilizer of claim 1, further comprising:
four support rods connected to the housing and spaced apart from each other at predetermined intervals.

6. The plasma and VHP combined airborne sterilizer of claim 5, each of the four support rods is a plasma and VHP combined airborne sterilizer characterized by an openable cover at the top and bottom of the support rod.

7. The plasma and VHP combined airborne sterilizer of claim 2, wherein the control unit to change from the first operation mode to the second operation mode when an external humidity amount is greater than a threshold value.

8. The plasma and VHP combined airborne sterilizer of claim 5, the diameter of the support rod is the same as the diameter of a discharge part.

9. The plasma and VHP combined airborne sterilizer of claim 1, wherein the first plasma module, a second plasma module, and a third plasma module are spaced apart from each other at a predetermined interval and placed in a ‘n’ shape, and the first plasma module and the second plasma module face each other.

10. A method of operating a plasma and vaporized hydrogen peroxide (VHP) combined airborne sterilizer, the method comprising:
emitting, with a Hydrogen peroxide emission stage, vaporized hydrogen peroxide particles of a predetermined size,
inhaling external air and the vaporized hydrogen peroxide particles with an external air and hydrogen peroxide inhalation stage, and
generating, with plasma modules, plasma ions for the vaporized hydrogen peroxide from the external air and hydrogen peroxide inhalation stage.

11. The method of claim 10 further comprising:
a humidity monitoring operation of determining whether a humidity measured in the humidity monitoring operation is above a set humidity by measuring the humidity due to hydrogen peroxide vaporized in the hydrogen peroxide emission stage.
